(19) United States
(12) Patent Application Publication
(10) Pub. No.: US 2004/0213698 A1
(43) Pub. Date: Oct. 28, 2004

(54) Electrochemical method and apparatus for generating a mouth rinse

(76) Inventors: Charles L.K. Tennakoon, College Station, TX (US); Rattaya C. Yalamanchili, College Station, TX (US); Elizabeth I. McGrew, Bryan, TX (US)

Correspondence Address: STREETS & STEELE, 13831 NORTHWEST FREEWAY, SUITE 355, HOUSTON, TX, 77040, US

(21) Appl. No.: 10/424,578

(22) Filed: Apr. 25, 2003

(51) Int. Cl. A61L 2/00

(52) U.S. Cl. 422/37; 422/29; 422/22; 422/186; 422/293

(57) ABSTRACT

An apparatus and method for electrochemically generating and using an aqueous solution containing chlorine dichloride and a metal hypochlorite, such as sodium hypochlorite. The aqueous solution provides a mouth rinse for oral hygiene or a sterilizing solution for surgical or dental tools or for treating drinking water. The generator comprises a chamber, an anode and a cathode disposed for fluid communication with a reservoir, and an electrical power source for applying a voltage between the anode and the cathode. The reservoir contains an electrolyte having a metal chlorite and, optionally, a metal chloride, citric acid, sweeteners, flavorings and combinations thereof. The anode is preferably a dimensionally stable anode coated with RuO₂, IrO₂ or combinations thereof. The cathode may be made of titanium substrate coated with platinum. The apparatus and method generate an aqueous solution containing chlorine dioxide that may be used immediately for oral hygiene.

### BACKGROUND OF THE INVENTION

1. Field of the Invention

The present invention relates to electrochemical methods and apparatus for generating a disinfectant or sterilant solution, such as a mouth rinse.

2. Description of the Related Art

Oral malodor, plaque, gingivitis, periodontal disease, and discoloration of the teeth are all undesirable conditions that affect many people. One significant cause of malodor of the oral cavity, also known as halitosis or bad breath, is the presence of anaerobic bacteria at the back of the tongue. These bacteria generate volatile sulfur compounds by degrading sulfur-containing amino acids present in the mouth, and the exhalation of these volatile sulfur compounds is perceived as bad breath.

Another source of volatile sulfur compounds is the degradation of epithelial cells and bacteria within the oral cavity. Specifically, the polypeptide chains of the epithelial cell walls are composed of a series of amino acids including cysteine and methionine, which contain sulfur side chains. The death of microorganisms or epithelial cells results in degradation of the polypeptide chains into their amino acid components, especially cysteine and methionine. Cysteine and methionine are precursors to the formation of volatile sulfur compounds. Furthermore, a person with gingivitis or periodontal disease may have increased oral malodor from disintegrated epithelial cells because the epithelial cells turn over faster if inflammation is present. These larger numbers of dead epithelial cells remaining in the oral cavity degrade into the malodorous compounds.

The volatile sulfur compounds that are generally recognized as being the cause of oral malodor and that are produced by the anaerobic bacteria and by the degradation of epithelial cells and bacteria, include hydrogen sulfide (H₂S), methyl mercaptan (CH₂—S—H) and dimethyl sulfide (CH₂—S—CH₃). Significantly, not only do these compounds cause oral malodor, these compounds also alter the epithelial barrier, which permits penetration of the barrier by antigenic substances, such as bacterial toxins, bacteria and viruses, into the underlying basal lamina and connective tissue.

Periodontal disease is also an undesirable condition that has widespread occurrence. Periodontal disease is a major cause of tooth loss in adults, beginning as early as age 12. Periodontal disease affects the periodontum, which is the investing and supporting tissues surrounding a tooth (i.e., the periodontal ligament, the gingiva, and the alveolar bone). Gingivitis and periodontitis are inflammatory disorders of the gingiva and the deeper periodontal tissues, respectively.

It is well accepted that periodontal disease is associated with the accumulation of plaque on the teeth. Plaque is formed when the teeth are coated with a salivary proteanaceous material (pellicle) to which streptococci and other bacteria adheres. Gingivitis occurs from the dental plaque, and periodontitis is caused by the infection spreading to the periodontal pocket or space between the gingiva and the tooth root. It is thought that gingivitis and periodontitis, and the bacteria associated with these inflammatory disorders, may further cause cardiovascular and pulmonary diseases.

It has been recognized that mouth washes, mouth rinses, toothpastes and other oral hygiene products are useful for decreasing the number of bacteria in the oral cavity and further useful in breaking down the volatile sulfur compounds that are found in the oral cavity to less malodorous material. Two compounds have been found to be effective both as an oxidizer capable of breaking down the volatile sulfur compounds and as a bactericide. These are chlorine dioxide and sodium hypochlorite.

Sodium hypochlorite is the active ingredient of common household bleach. Chlorine dioxide is a very strong oxidant and is known as a broad-spectrum antimicrobial agent as well as being an effective bleach and deodorant, and is used industrially in that capacity. Molecular chlorine dioxide (ClO₂) is a water soluble gas with a high oxidation potential. Because of this high oxidative tendency, chlorine dioxide is readily able to oxidize the volatile sulfur compounds causing the mercaptans, sulfides and disulfides to lose most, if not all, their malodor properties upon being oxidized.

Richter, in U.S. Pat. No. 5,738,840, issued Apr. 14, 1998, discloses an oral rinse method for treating halitosis. In the disclosed method, a kit for preparing the oral rinse treatment contains a buffered aqueous sodium chlorite solution in one container and aqueous sodium hypochlorite in another container, which is also buffered. The two solutions are mixed just prior to use for the generation of a solution of dissolved chlorine dioxide.

Whitt, et. al., in U.S. Pat. No. 6,251,372 B1, issued Jun. 26, 2001, discloses a mouth rinse having a low concentration of dissolved chlorine dioxide made by mixing two solutions together; one solution containing sodium chlorite buffered at pH 10 and the other containing another solution which, when mixed in 1:1 volume ratio, gives a pH of approximately 8.5 to 9. The same patent also describes another single-phase mouthwash containing sodium chlorite, sodium carbonate, and sodium bicarbonate in water.

Electrochemical reactors are well known in general and are used in many different industries. For example, electrolysis of molten sodium chloride provides metallic sodium and chlorine gas and the electrolysis of water provides hydrogen and oxygen. Electrochemical cells in which a chemical reaction is forced by adding electrical energy are called electrolytic cells. Central to the operation of any cell is the occurrence of oxidation and reduction reactions that produce or consume electrons. These reactions take place at electrode/solution or electrode/gas phase interfaces, where the electrodes must be good electronic conductors.

In operation, a cell is connected to an external load or to an external voltage source, and electrons transfer electric charge between the anode and the cathode through the external circuit. To complete the electric circuit through the cell, an additional mechanism must exist for internal charge transfer. This is provided by one or more electrolytes, which support charge transfer by ionic conduction, but they must be poor electronic conductors to prevent internal short-circuiting of the cell.

The simplest electrochemical cell consists of at least two electrodes and one or more electrolytes. The electrode at which the electron producing oxidation reaction occurs is the anode. The electrode at which an electron consuming reduction reaction occurs is called the cathode. The direction of the electron flow in the external circuit is always from the anode to the cathode.

The efficacy of increasing oral hygiene with oral hygiene products that contain chlorine dioxide and hypochlorite, such as sodium hypochlorite, is well known but because of the instability of chlorine dioxide, there are no convenient means for allowing consumers to use such products daily. It would be advantageous if chlorine dioxide could be produced quickly and easily in a sufficient quantity for immediate use without requiring storage for any significant period.

Therefore, there is a need for devices and methods that will provide a highly effective oral hygiene treatment. Preferably, the devices and methods will result in decreased bacterial activity in the oral cavity, a reduction in plaque formation and a reduction in oral malodor.

### SUMMARY OF THE INVENTION

One embodiment provides an apparatus for electrochemically generating chlorine dioxide for use as a mouthwash or as a sterilant. The apparatus comprises a chamber or case containing an anode and a cathode that are disposed for fluid communication with an electrolyte contained in a reservoir. The apparatus further comprises an electrical power source for applying a voltage or current between the anode and the cathode. Preferably, the anode, the cathode, and the electrical power source are all contained within the case.

The anode electrode will typically be comprised of a metal. The metal may include, without limitation, titanium, platinum, and combinations thereof. Preferably, the anode has a titanium substrate coated with an oxide selected from RuO₂, IrO₂ and combinations thereof. The oxide coating may further be mixed with materials selected from cobalt, iron, bismuth and combinations thereof to enhance the catalytic activity of the oxide coating.

The cathode electrode will typically be comprised of a metal or graphite. Acceptable metals may include, without limitation, titanium, platinum, graphite, steel, gold, iron, silver, tin, nickel and combinations thereof. Preferably, the cathode has a titanium substrate coated with a metal, for example a metal selected from platinum, stainless steel, nickel, gold and combinations thereof.

The electrolyte includes an aqueous solution, containing a metal chlorite, such as sodium chlorite. Other components of the electrolyte may include, without limitation, sodium chloride, an organic acid, citric acid, a flavoring, a buffer and combinations thereof. Other additives that do not interfere with generation or use of chlorine dioxide may also be included in the electrolyte solution. The concentration of sodium chlorite in the electrolyte may be between about 0.01 g/L and about 6 g/L, preferably between about 0.25 g/L and about 1 g/L, and most preferably between about 0.5 g/L and about 0.6 g/L. If the electrolyte includes sodium chloride, the molar ratio of the sodium chloride to the sodium chlorite may be between about 0.1 and 10, preferably between about 2 and about 9, and most preferably between about 6 and about 8.

Preferably, the distance separating the anode and the cathode (the electrodes) will be between about 0.3 mm and about 10 mm, and most preferably between about 0.5 and about 1 mm. The cathode and the anode may have any shape or configuration, specifically including a rod, a plate, a wire, a spiral, a Swiss-roll and combinations thereof. Whereas the anode and the cathode may each have any suitable dimension, in a preferred embodiment the anode and the cathode will each measure between about 0.5 cm² and about 40 cm², more preferably between about 1 cm² and about 10 cm^{2,} or even more preferably between about 1 cm² and about 5 cm². It should also be recognized that any number of anodes and cathodes may be used in accordance with the invention.

The voltage applied across the electrodes is preferably between about 1.3 V and about 5 V, most preferably between about 2 V and about 3 V. The power source that provides the voltage may be one or more batteries, fuel cells, or a standard electrical wall outlet with a transducer. The current density imparted upon the electrodes is preferably between about 5 mA/cm² and about 100 mA/cm², more preferably between about 5 mA/cm² and about 30 mA/cm², and most preferably between about 10 mA/cm² and about 20 mA/cm².

The apparatus may further comprise a pellet storage compartment, wherein the pellets contain premeasured amounts of the ingredients of the electrolyte, such as: the reactants sodium chloride, sodium chlorite, or combinations thereof; other additives like citric acid, a buffer, or combinations thereof; or combinations of reactants and other additives.

Another embodiment provides a small portable mouthwash generator having a case, an anode and a cathode disposed for fluid communication with a reservoir, an electrical power source for applying a voltage or current between the anode and the cathode, wherein the anode, the cathode and the electrical power source are contained within the case. The reservoir must contain an electrolyte, wherein the electrolyte comprises sodium chlorite. Chlorine dioxide is produced by electrolytic reaction when a voltage or current is applied across the electrodes immersed in the electrolyte. The electrolyte may comprise other ingredients or additives selected from, for example, sodium chloride, an organic acid, a flavoring, a buffer and combinations thereof.

In a further embodiment, a mouth irrigator having a reservoir is combined with an anode and a cathode of a mouthwash generator. The anode and the cathode are disposed for fluid communication with an electrolyte within the reservoir. The mouth irrigator further provides an applicator for delivering a fluid from the reservoir to the user's mouth, and an electrical power source for applying a voltage between the anode and the cathode.

In yet another embodiment, a sterilizer is provided having a sterilization chamber, an anode and a cathode. The anode and the cathode are disposed for fluid communication with an electrolyte chamber containing an electrolyte comprising sodium chlorite. An electrical power source is provided for applying a voltage between the anode and the cathode to generate chlorine dioxide by electrochemical reaction.

The sterilization chamber and the electrolyte chamber may be the same chamber or they may be chambers separated in part or totally by a gas permeable membrane. The gas permeable membrane may be a material selected from PTFE (ethylene tetrafluoride resin), PFA (ethylene tetrafluoride-perfluoroalkoxyethylene copolymer resin), PVDF (vinylidene fluoride resin), FEP (ethylene tetrafluoride-propylene hexafluoride copolymer resin), ETFE (ethylene tetrafluoride-ethylene copolymer resin), and combinations thereof. The pore size of the gas permeable membrane may be between about 0.01 μm and about 10 μm, or between about 0.1 μm and about 2 μm.

Optionally, the sterilization chamber may have means for communicating with a vacuum source for evacuating the sterilization chamber after instruments to be sterilized have been placed within the chamber.

A still further embodiment provides a method of disinfecting, the method comprising preparing an aqueous sodium chlorite solution, applying a voltage between an anode and cathode within the solution to generate chlorine dioxide gas within the aqueous liquid. The method produces an aqueous solution comprising dissolved chlorine dioxide gas. Beneficially, a contaminated surface may be contacted with the aqueous chlorine dioxide gas-containing solution within a period of about 30 minutes after generating the chlorine dioxide gas. The aqueous liquid is preferably an electrolyte. The method may further include dissolving an additive such as sodium chloride, flavorings, a buffer, a sweetener, citric acid or combinations thereof into the aqueous liquid before, during, or after generating the chlorine dioxide gas. If additives are added to the solution, they are most preferably provided during the step of preparing the aqueous sodium chlorite solution, such as by dissolving a single pellet that includes the sodium chlorite as well as the additives.

The foregoing and other features and advantages of the invention will be apparent from the following more particular description of a preferred embodiment of the invention, as illustrated in the accompanying drawing wherein like reference numbers represent like parts of the invention.

### BRIEF DESCRIPTION OF THE DRAWINGS

FIG. 1 is a schematic view of an electrochemical cell in accordance with the present invention.

FIG. 2 is a cut away view of the “pen” mouthwash generator in accordance with the present invention.

FIG. 3 is a perspective view of a modified mouth irrigator that is coupled with a mouthwash generator in accordance with the present invention.

FIGS. 4A-B is a cut away view of the medical instrument sterilizer in accordance with the present invention and a detail of a spiral electrode.

FIGS. 5A-C are perspective views of a medical instrument sterilizer having flexible walls in accordance with the present invention.

FIG. 6A-D are perspective views of examples of electrode assemblies that may be used in conjunction with the present invention.

FIG. 7 is an exploded view of a single cell chlorine dioxide/hypochlorite generator.

FIG. 8 is a graph showing chlorine dioxide generated in the cell shown in FIG. 7 over time.

FIGS. 9A-B are graphs showing chlorine dioxide and hypochlorous acid and hypochlorite ion generation in the cell shown in FIG. 7 over time.

FIG. 10A-B are graphs showing chlorine dioxide and hypochlorous acid and hypochlorite ion generation in a miniaturized cell at different currents.

### DETAILED DESCRIPTION OF PREFERRED EMBODIMENTS

The present invention provides an apparatus and methods for generating and using an aqueous solution containing chlorine dioxide and a hypochlorite, such as sodium hypochlorite. The solution product may be used as a mouth rinse for oral hygiene or, for example, as a sterilizing solution for surgical or dental tools or for treating drinking water. The generator comprises a chamber, an anode and a cathode disposed for fluid communication with a reservoir, and an electrical power source for applying a voltage between the anode and the cathode.

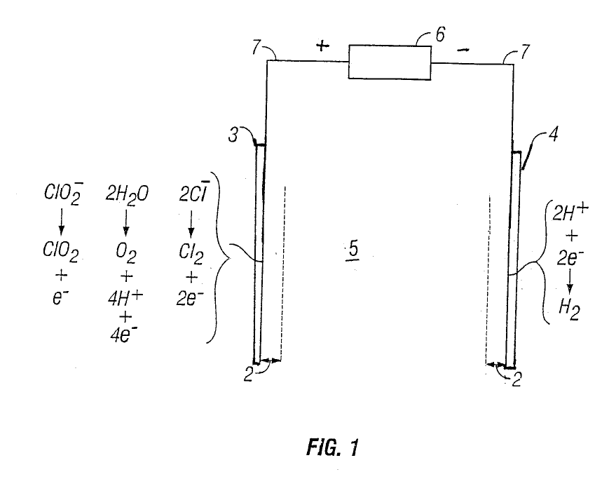

Chlorine dioxide and a hypochlorite may be generated in an electrochemical cell containing, for example, sodium chlorite and sodium chloride dissolved in an aqueous solution as an electrolyte. Sodium chlorite (NaClO₂) and sodium chloride (NaCl) dissociate in the aqueous solution to Na⁺, ClO₂⁻, and Cl⁻. FIG. 1 is a schematic view of an electrochemical cell in accordance with the present invention. An anode 3 and a cathode 4 are immersed in an electrolyte 5. The electrolyte is an aqueous solution of sodium chlorite and sodium chloride. A power supply 6 provides a voltage between the anode 3 and the cathode 4 through a conductor or wire 7. A diffusion layer 2 exists at both the anode and the cathode.

At the anode 3, the following oxidation reactions occur in the formation of chlorine dioxide (ClO₂):

ClO₂⁻→ClO₂+e⁻ (Eq. 1)

Cl⁻→Cl₂+2e⁻ (Eq.2)

2H₂O→O₂+4H⁺+4e⁻ (Eq. 3)

In Equation 1, the ClO₂⁻ that was formed from the dissociation of the dissolved sodium chlorite is oxidized at the anode to yield chlorine dioxide and an electron. In Equation 2, the Cl⁻ that was formed from the dissociation of the dissolved sodium chloride is oxidized to yield chlorine gas and two electrons. Finally, some of the water is oxidized to yield oxygen gas, four electrons and four protons. The liberated protons are transported through the electrolyte to the cathode 4. The electrons liberated at the anode 3 are transported to the cathode 4 through the conductor 7 that connects the anode 3 and the cathode 4 to the power supply 6.

At the cathode 4, the following reaction occurs:

2H⁺+2e⁻→H₂ (Eq. 4)

In Equation 4, the protons that were conducted through the electrolyte from the anode 3 to the cathode 4, and the electrons that were conducted to the cathode 4 from the anode 3 through the wire 7 are combined to form hydrogen gas.

Additional chlorine dioxide is generated by reacting some of the chlorine gas, which was electrochemically formed as shown in Equation 2, with some of the sodium chlorite from the electrolyte, according to the following reaction:

NaClO₂+Cl₂→ClO₂+NaCl (Eq. 5)

To form a hypochlorite, some of the electrochemically formed chlorine formed as shown in Equation 2 reacts under the pH conditions that exist in the bulk of the solution to generate hypochlorite (OCl⁻) according to the following reaction:

Cl₂+2OH⁻→Cl⁻+OCl⁻+H₂O (Eq. 6)

Therefore, as shown in Equations 1 through 6, an electrochemical cell having an electrolyte consisting of an aqueous solution of sodium chloride and sodium chlorite can produce chlorine dioxide and hypochlorite. The concentrations of the chlorine dioxide that is generated may be between about 1 ppm and about 500 ppm. Typically, the concentration may be between about 5 ppm and about 60 ppm. Preferably, for a mouth rinse, the concentration of chlorine dioxide may be between about 10 ppm and about 30 ppm. Likewise, the concentration of the hypochlorite may be between about 1 ppm and about 6000 ppm, but more typically the concentration may be between about 5 ppm and about 100 ppm and preferably, for a mouth rinse, my be between about 10 ppm and about 30 ppm.

The molar ratio of the concentration of the sodium chloride to the sodium chlorite in the electrolytic aqueous solution may range from between about 0.1 and 10. Typically, the molar ratio may range from between about 2 and about 9 and preferably the molar ratio will be between about 6 and about 8. The concentration of the sodium chlorite in the electrolytic aqueous solution may range from between about 0.01 g/L to about 6 g/L. More typically, the range may be between about 0.25 g/L and about 1 g/L. Preferably, the range of the concentration of the sodium chlorite in the aqueous solution may range from between about 0.5 g/L and about 0.6 g/L. Alternatively, there may be no sodium chloride contained in the electrolytic solution. Other metal chlorides and metal chlorites may be used in the same proportion. For example, potassium chloride and/or potassium chlorite may be used as alternatives to the sodium chloride or sodium chlorite or both.

The electrodes may be made of many known materials. The anode is preferably a dimensionally stable anode, an electrode having a titanium substrate coated with an oxide such as, for example, RuO₂, IrO₂, or mixtures of these or other transition metal oxides. Additionally, one or more dopants, such as cobalt, iron, bismuth, or combinations thereof, may be added to the oxides to enhance their catalytic activity. Alternatively, the anode may be made of titanium, platinum, or titanium plated with platinum.

The anode substrate may optionally be coated with oxides of ruthenium, iridium or combinations of these or other transition metal oxides by electroplating. The method for coating the substrate includes preparing an aqueous solution of halide compounds containing ruthenium, iridium or other transition metals. Examples of suitable compounds include, for example, K₂RuCl₆, K₃RuCl₆, K₃RuBr₆, K₂RuBr₆, K₂IrCl₆, K₃IrCl₆, K₃RuBr₆, and K₃IrBr₆. To increase the solubility, a dicarboxylic acid, for example, oxalic acid or succenic acid, may be added to the aqueous solution so that soluble complexes are formed with the dicarboxylic ligands. Carbonates or hydroxides may be added to the aqueous solution to raise the pH above 7. The pH may be between about 7 and about 11, preferably between about 9 and about 10. Suitable carbonates or hydroxides include, for example, sodium carbonate, potassium carbonate, sodium hydroxide, sodium carbonate and combinations thereof.

The halide compounds are added to the aqueous solution to form a concentration between about 0.2 g and about 1 g per 100 ml of water. Alternatively, the halide compounds may be added to the aqueous solution to form a concentration of between about 0.3 g and about 0.5 g per 100 ml of water.

The dicarboxylic acid is added to the aqueous solution to form aconcentration between about 0.1 g and about 5 g per 100 ml of water. The hydroxide or carbonate is added to raise the pH to a level that promotes the formation of the halide complexes with the dicarboxylic acid ligands.

The aqueous solution is then held or stored for a period at a temperature between about 25° C. and about 45° C. or between about 30° C. and about 40° C. During the period, usually about four days, the solution will undergo several color changes. For example, when using potassium hexachloroiridate in the aqueous solution, the color of the solution will change from greenish, to brown, to clear and then to purple. When using potassium hexachlororuthenate, the color of the solution changes from maroon, to green, to brown, and then to black.

After the color changes have been completed during the hold period, the substrate to be coated may be immersed in the solution for electroplating. The substrate to be coated is immersed in the solution as an anode and a cathode also is immersed in the solution. A current density of between about 0.1 mA/cm² and about 1 mA/cm² is applied to coat the substrate with the oxides in the electroplating process. The cathode may be of any suitable material, such as a titanium substrate coated with stainless steel, platinum, nickel, gold, and combinations thereof. Other suitable materials include, for example, titanium, platinum, graphite, steel, gold, iron, silver and tin.

The cathode may be made of many different known materials and a suitable material should be non-corrosive in the aqueous electrolyte. The cathode may be a titanium substrate coated with stainless steel, platinum, nickel, gold, and combinations thereof. The cathode is preferably made of a platinum-coated titanium substrate. Examples of alternative cathode materials include titanium, platinum, graphite, steel, gold, iron, silver and tin.

To minimize power requirements, it is desirable to place the two electrodes as closely together as possible without touching. The gap between the electrodes will preferably be between about 0.3 mm and about 10 mm. More preferably, the gap between the electrodes will measure between about 0.5 mm and about 1 mm. The shape of the electrodes is not limited and may include, for example, rods, plates, wires, spirals, Swiss-roll or combinations thereof. When both electrodes are spirals or Swiss-rolls, they may preferably be wound with faces adjacent to each other over the entire length of the spiral. In applications that are not highly concerned with power requirements, then the distance between the electrodes may be much greater.

The voltage across the electrodes may range between about 1.3 V and about 5 V. Preferably, the voltage across the electrodes ranges between about 2 V and about 3 V but the voltage requirement is set as being the voltage required to maintain the required current density and may therefore be more or less than the stated ranges. Current density will typically be between about 5 mA/cm² and about 100 mA/cm², preferably between about 5 mA/cm² and about 30 mA/cm², and most preferably between about 10 mA/cm² and about 20 mA/cm². It should be recognized that the voltage or current may be controlled in various manners, specifically including, without limitation, constant or variable voltage, or constant or variable current, or constant or variable power.

The pH of the electrolyte will preferably be between about 5 to about 11, more preferably between about 6 and about 10, and most preferably between about 6 and about 8 pH. Additives known in the art may be added to the electrolyte to achieve a desired pH. For example, an organic acid, such as citric acid, may be added to adjust the pH of the electrolyte to a desired level.

If desired, the chlorine dioxide formed in the electrolytic aqueous phase may be separated from the electrolytic aqueous phase and accumulated in a vapor space. A gas permeable membrane may separate a vapor space from the electrolytic aqueous phase. Once enough chlorine dioxide has been generated to saturate the electrolytic aqueous solution, any additional chorine dioxide that is generated will not remain dissolved in the electrolyte but will disengage from the solution and pass through the gas permeable membrane into the vapor space on the opposite side of the membrane. The chlorine dioxide accumulated in the vapor space may then be used as a sterilant.

There is no particular restriction on the nature of the permeable membrane to be used to separate the vapor space from the electrolytic aqueous phase. The membrane may be formed with, for example, PTFE (ethylene tetrafluoride resin), PFA (ethylene tetrafluoride-perfluoroalkoxyethylene copolymer resin), PVDF (vinylidene fluoride resin), FEP (ethylene tetrafluoride-propylene hexafluoride copolymer resin), or ETFE (ethylene tetrafluoride-ethylene copolymer resin). The pore size of the membrane may be selected such that water does not permeate through the membrane used, and the mean pore size is preferably between about 0.01 μm and about 10 μm, and more preferably between about 0.1 μm and about 2 μm thick.

In one embodiment, a mouthwash generator sized to accommodate two AAA batteries as a power source, may include an electrolytic cell for generating a mouthwash in accordance with the present invention. This portable mouthwash generator may be used easily and quickly to generate a mouthwash containing chlorine dioxide and, optionally, hypochlorite for personal immediate or prompt use, generally at the point-of-generation. A small electrode assembly having a cathode and an anode connected to a power source is contained within the case of the portable mouthwash generator. For a portable generator, the power source is preferably one or more batteries contained within the case of the mouthwash generator. The electrode assembly is located behind a grill formed at one end of the case. An electrochemical cell may therefore be completed by immersing the electrode assembly contained within the mouthwash generator into a container of electrolyte. With a voltage applied across the immersed electrodes, the electrochemical reactions proceed and chlorine dioxide and, optionally, hypochlorite are generated in the aqueous electrolyte, thereby forming the mouthwash.

The electrolyte used with the mouthwash generator is preferably an aqueous solution having a metal chloride and a metal chlorite, such as sodium chloride and sodium chlorite. Optionally, citric acid or another organic acid may be added to the electrolyte to adjust the pH to a desired level. Other additives may include flavorings, sweeteners, buffers and combinations thereof. Desired amounts of metal chloride, metal chlorite, citric acid and other desired additives may be added to a container of water to form the aqueous electrolyte. Preferably, premeasured amounts of these desired components may be formed into pellets or packaged in small bags so that these premeasured amounts may then be added to a container (generally establishing the reservoir), such as a water glass, containing a measured amount of water.

Optionally, the mouthwash generator may further include a pellet storage compartment, wherein pellets comprising sodium chlorite, sodium chloride and other desirable ingredients may be stored. Of course, other metal chlorites or metal chlorides may be used. These pellets may be manufactured with the desired molar ratio of each ingredient so that when one or more pellets are dissolved in a measured amount of water, the resulting aqueous electrolyte solution contains the desired amount of each ingredient for the electrolyte and the resulting mouthwash. Preferably, a volume of about 20 ml of mouthwash is generated for each use, although it is anticipated that the volume might be between about 5 ml and about 500 ml or between about 10 ml and about 30 ml.

In another embodiment, a mouthwash generator in accordance with the present invention may be coupled with a mouth irrigator or oral irrigator. Mouth irrigators are oral hygiene devices that pulse powerful jets of water from an applicator onto the teeth and gums to remove food particles and plaque buildup. Existing mouth irrigators typically already include a reservoir and a pump for pumping the water from the reservoir into the mouth at a high velocity.

The mouthwash generator that is coupled to the mouth irrigator includes anode and cathode electrodes that must be immersed in an electrolyte contained within the reservoir of the mouth irrigator. In one embodiment, a removable electrode assembly containing the electrodes may be placed in the reservoir whenever a mouthwash generation is desired. Alternatively, the electrodes may be permanently placed or installed in the reservoir so that the electrode assembly is immersed whenever the reservoir is filled. The removable electrode assembly is preferred for after-market products that could be used to adapt an existing mouth irrigator with the mouthwash generator. However, installing permanent electrodes in the reservoir as original equipment is preferred for a new mouth irrigator having the feature of a mouthwash generator. In another alternative, since the mouth irrigator has a pump, the electrodes may be immersed in a stream that flows from the reservoir, through the pump, past the electrodes, and then either back to the reservoir or for delivery to the nozzle.

To generate an electrolytic aqueous solution for the mouthwash generator, a measured amount of ingredients selected from sodium chloride, sodium chlorite, citric acid, flavorings, sweeteners, buffers and combinations thereof may be added to water in the reservoir. Alternatively, other metal chlorides or metal chlorites may be included to replace or enhance the sodium forms. When voltage is applied across the electrodes from a power supply, preferably the power supply of the mouth irrigator, chlorine dioxide and hypochlorite (depending upon the ingredients used) are formed in solution with the aqueous electrolyte solution and a mouthwash is thereby generated in the reservoir of the mouth irrigator. This mouthwash may then be used to irrigate the mouth and to rinse the mouth and gargle as desired. Typically, about 500 ml of mouthwash will typically be generated for a mouth irrigator although the volume of mouthwash in the reservoir may vary between about 100 ml and about 1000 ml or preferably between about 400 ml to about 600 ml of mouthwash containing chlorine dioxide and hypochlorite.

In another embodiment, a sterilization unit may include an electrolytic cell for generating a sterilant in accordance with the present invention. The sterilization unit may be used to sterilize, for example, surgical or dental instruments in a doctor's office or in the field where other sterilization devices may not be available. The sterilization device may sterilize the instruments by generating a sterilizing solution containing chlorine dioxide and hypochlorite in which the instruments are immersed. Alternatively, the instruments may be contained within a separate sterilization chamber that is separated from the electrolyte chamber by a gas permeable membrane. Chlorine dioxide generated at the electrode assembly immersed in the electrolyte may then pass through the gas permeable membrane into the sterilization chamber that contains the instruments to be sterilized. Separating the sterilization chamber from the electrolyte chamber and thereby sterilizing the instruments with the gas that passes through the gas permeable membrane is preferred because then, the sterilized instruments are dry and ready for use when the instruments are removed from the sterilization chamber.

Swiss-roll wound electrodes are preferred, but not required, for the sterilization unit due to the limited space that is available for the electrodes in the electrolyte chamber. Swiss-roll wound electrodes include a cathode and anode that are each formed from a flat strip and then wound together in a spiral, while still maintaining a constant gap between the faces of the anode and the cathode. Each of the electrodes may then be connected to a power source, such as one or more batteries or to a standard electrical outlet through a transducer.

The chambers of the sterilization unit may be formed with rigid walls, such as plastic or metal, or the walls may be formed with flexible walls, such as a sealable plastic bag. A preferred embodiment includes a sterilization chamber made from a plastic vacuum bag.

A chamber made of rigid walls preferably includes a gas-tight lid that may be removed for placing the instruments to be sterilized into a separate sterilization chamber or alternatively, into a single combined sterilization and electrolyte chamber. A chamber made with flexible walls, preferably a plastic vacuum bag, may be sealed by heat or alternatively, by clamping or zipping the plastic bag shut after the instruments are placed into the bag. Preferably, the sterilization unit includes a sealable plastic bag, such as a plastic vacuum bag, for use as the sterilization chamber. Many vacuum bags, for example, are made of a nylon/polyethylene combination material, normally having a thickness of between about 2 mil and 7 mil. Nylon is preferably used in the bag construction to provide a longer vacuum life because without the nylon, air from the outside atmosphere will refill the bag in just a few hours. The bag may be sealed with heat or alternatively, the bag may be clamped or zipped closed, after the instruments to be sterilized are placed within the bag. The sterilization unit preferably includes a separate electrolyte chamber separated from the sterilization chamber by a gas permeable membrane. The electrodes and/or the permeable membrane may be protected from being damaged by the instruments with a slotted plastic barrier or similar device.

To form the electrolyte to be used with the sterilization unit, a measured amount of ingredients selected, for example, from sodium chloride, sodium chlorite, citric acid or other organic acid, buffers and combinations thereof may be added to the electrolyte chamber. Alternatively, other metal chlorides or metal chlorites may be added. Preferably, the ingredients are premeasured and packaged in separate packets or compressed into one or more pellets so that a prescribed number of packets or pellets may be added to the known volume of water to be added to the electrolyte chamber. The electrolyte chamber may then be filled with water to generate an aqueous electrolyte solution. When voltage is applied across the electrodes from the power supply, chlorine dioxide and hypochlorite is generated in accordance with the present invention, generating a sterilizing solution in the electrolytic chamber. If the sterilizing chamber is separated from the electrolytic chamber by a gas permeable membrane, then the chlorine dioxide that is generated passes through the membrane into the sterilization chamber. Electrical power is applied to the electrodes for a set period to generate sufficient chlorine dioxide for sterilizing the instruments. The instruments will remain sterilized until the point of use as long as they remain within the sterilization chamber.

In a preferred embodiment using a sterilization chamber separated from the electrolyte chamber by a gas permeable membrane, the sterilization chamber is evacuated by a vacuum pump through a valved connection for that purpose. By evacuating the sterilization chamber prior to generating the chlorine dioxide, a much higher concentration of the sterilant chlorine dioxide may be achieved in the sterilization chamber since the generated chlorine dioxide is not diluted with the removed air. Optionally, the valved connection may be attached to a vacuum pump prior to removing the sterilized instruments from the sterilization chamber, so that the chlorine dioxide may be evacuated from the sterilization chamber and vented to a safe location. Alternatively, the chlorine dioxide being evacuated from the sterilization chamber may pass through a transparent tube exposed to a light source so that the chlorine dioxide decomposes to harmless components.

When evacuating the sterilization chamber with a vacuum pump, the gas permeable membrane must be protected from being damaged by pressure differential across the membrane. A protective cap may therefore be placed over the membrane to seal the membrane from atmospheric pressure while a vacuum is being pulled on the opposite side.

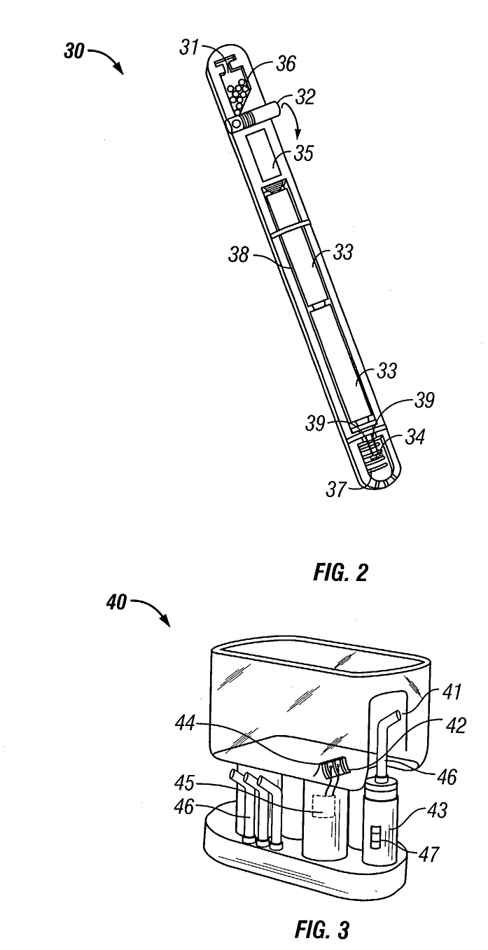

FIG. 2 is a cut away view of a handheld or “pen”-sized mouthwash generator in accordance with the present invention. The mouthwash generator 30 in the form of a “pen” is shown as a self-contained mechanical and electronic unit. A pellet storage compartment 31 stores pellets 36 containing the ingredients that may be dissolved in a measured amount of water to form an electrolytic aqueous solution. The pellets 36 contain premeasured amounts of the ingredients selected from sodium chloride, sodium chlorite, flavorings, citric acid, buffers and combinations thereof. Batteries 33, contained in the battery compartment 38 having a removable cover (not shown) for battery replacement, provide the power source required by the electrodes 34. The electrodes 34 include a cathode electrode and an anode electrode, each connected to an electronic control board 35 by a conductor 39. The batteries 33 are also connected to the electronic control board 35 so that a completed circuit is formed between the anode and the cathode electrodes 34. A grill 37 formed in the case of the mouthwash generator 30 allow the electrodes 34 to be immersed in the electrolyte when the electrode end of the mouthwash generator 30 is immersed in a container of electrolyte. It should be noted that the electrodes 34 are wetted when immersed in the electrolyte, but the battery compartment 38 is sealed against the electrolyte, even when the electrodes 34 are immersed.

When a user is ready to generate a quantity of mouthwash, the user removes a pellet 36 from the pellet storage chamber 31 through the pellet dispenser 32 and dissolves the pellet 36 in a measured amount of water contained in a container, such as a drinking glass. The water may be potable water or deionized water. In one design, twisting the halves of the mouthwash generator 30 with respect to one another activates the electrical system by moving the bottom half up until the battery 33 engages with its electrical contacts. In an alternative design, a switch may be used to complete the circuit with the batteries 33. When the mouthwash generator 10 is placed in the electrolyte to submerge the electrodes 34, and power is applied across the electrodes 34, the electrochemical generation of chorine dioxide and hypochlorite begins to form the mouthwash in the aqueous electrolyte solution.

FIG. 3 is a perspective view of a modified mouth irrigator that is coupled with a mouthwash generator in accordance with the present invention. The modified mouth irrigator 40 includes a water reservoir 41 in which the electrodes 42 may be permanently installed. The electrodes 42 include a cathode and anode electrode and means for connecting the electrodes to a power source, such as batteries or preferably, the power supply 45 of the mouth irrigator 40. The mouth irrigator 40 is typically supplied with power from a connection made through a standard electrical outlet. Water is pumped from the reservoir 40 through an applicator 43 into the mouth to remove food particles and plaque from the teeth and gums of the user. The applicator 43 includes a flow control selector 47 to control the quantity of water irrigating the mouth. Personal interchangeable applicator tubes 46 may be mounted onto the applicator 43 by different family members. The reservoir 41 typically holds between about 500 ml to about 1000 ml of water, which can be converted to a mouth rinse containing electrochemically generated chlorine dioxide and hypochlorite in accordance with the present invention.

After the reservoir 41 is filled to the desired level, the ingredients that are necessary to form the aqueous electrolyte solution are added to the reservoir 41. These ingredients may be selected from sodium chloride, sodium chlorite, flavorings, citric acid, buffers and combinations thereof. Preferably, the ingredients are premeasured and packaged in separate packets or compressed into a pellet so that a prescribed number of packets or pellets may be added to the measured amount of water in the reservoir 41. When voltage is applied across the electrodes 42 from the power supply, chlorine dioxide and hypochlorite are generated in accordance with the present invention, generating a mouth rinse in the reservoir 41, which may then be used to irrigate the mouth or to gargle. The power may be applied for a set period for the quantity of water in the reservoir 41 to ensure that a desired concentration of chlorine dioxide and hypochlorite are generated and dissolved in the mouth rinse prior to use. A timer may be set to remove the power automatically from the electrodes 42 at the preset period or the user may manually turn the power to the electrodes off after the prescribed period. More sophisticated embodiments may include one or more sensors capable of determining that a suitable rinse solution has been generated, such as pH sensors, conductivity sensors, product or by-product gas sensors, current sensors, liquid level sensors, and the like. A slotted plastic protector 44 may be used to cover the electrodes 42 to protect them from damage.

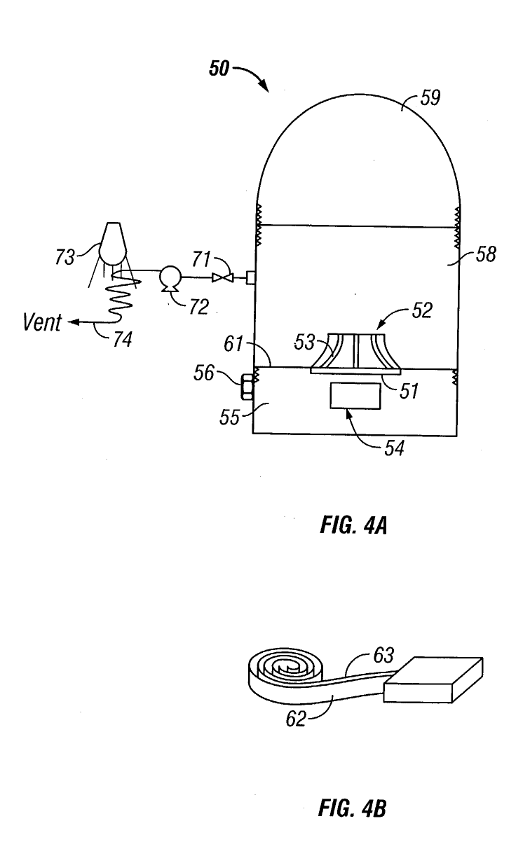

FIG. 4A-4B is a cut away view of the medical instrument sterilizer in accordance with the present invention and a detail of a spiral electrode. The instrument sterilizer 50 is made of a rigid material, such as a hard plastic or metal, and includes a sterilization chamber 58 that may be opened with a gas-tight lid 59. The gas-tight lid 59 is threaded and may be screwed into the top of the sterilization chamber 58. Medical instruments may be placed into the sterilization chamber 58 and then sealed into the sterilization chamber with the gas-tight lid 59. The floor 61 of the sterilization chamber 58 separates the sterilization chamber 58 from the electrolyte chamber 55. A gas permeable membrane 51 forms a part of the floor 61. A plastic protective cap 52 having slots 53 may be provided to cover and protect the gas permeable membrane 51 from puncture by the medical instruments being sterilized.

Water and other ingredients necessary to form the electrolytic aqueous solution may be poured or placed into the electrolyte chamber through a port having a removable seal cap 56. The ingredients that are dissolved in the water to form the aqueous electrolyte solution may be selected from sodium chloride, sodium chlorite, citric acid, buffers and combinations thereof. Preferably, the ingredients are premeasured and packaged in separate packets or compressed into a pellet so that a prescribed number of packets or pellets may be added to the known amount of water that the electrolyte chamber 55 may hold. The electrolyte chamber is preferably filled with the electrolyte so that the chamber contains little or no air. Alternatively, the ingredients that makeup the aqueous electrolyte solution may be mixed outside of the electrolyte chamber and then poured into the electrolyte chamber.

The electrodes 54 may be spiral wound electrodes as shown in FIG. 4B. The anode electrode 62 and the cathode electrode 63 are both wound into spirals so that the faces of each of the electrodes are preferably adjacent to and equidistant from each other along the length the cathode electrode 62 and anode electrode 63. When voltage is applied across the electrodes 54 from the power supply, chlorine dioxide is generated in accordance with the present invention, which passes through the permeable membrane 51 to enter the sterilization chamber 58. The power may be applied for a set period for the quantity of water in the electrolyte chamber 55 to ensure that a desired concentration of chlorine dioxide is generated. A timer may be set to remove the power automatically from the electrodes 54 at the preset period or the user may turn the power to the electrodes off after the prescribed period. A minimum chlorine dioxide gas concentration of between about 5 to about 10 ppm chlorine dioxide is preferred in the sterilization chamber 59.

Optionally, the sterilizing unit 50 may include a valve 71 that may be connected to a vacuum pump 72 for evacuating the sterilization chamber 58. The sterilization chamber 58 may be evacuated of air before the chlorine dioxide is generated to prevent the generated chlorine dioxide from being diluted by the air within the chamber 58. The sterilization chamber 58 may also be evacuated after the sterilization process to purge the chamber of the chlorine dioxide by venting it to a safe location or by exposing the chlorine dioxide to a light source 73 to decompose the chlorine dioxide. To prevent damage to the gas permeable membrane 51 from pressure differential when the sterilizing chamber 58 is placed under vacuum, the electrolyte reservoir 55 may be threaded and made removable from the sterilization chamber 58, thereby exposing the membrane 54. A threaded protective cap (not shown) may then be screwed onto the membrane support plate to protect the membrane 51 from pressure differential damage.

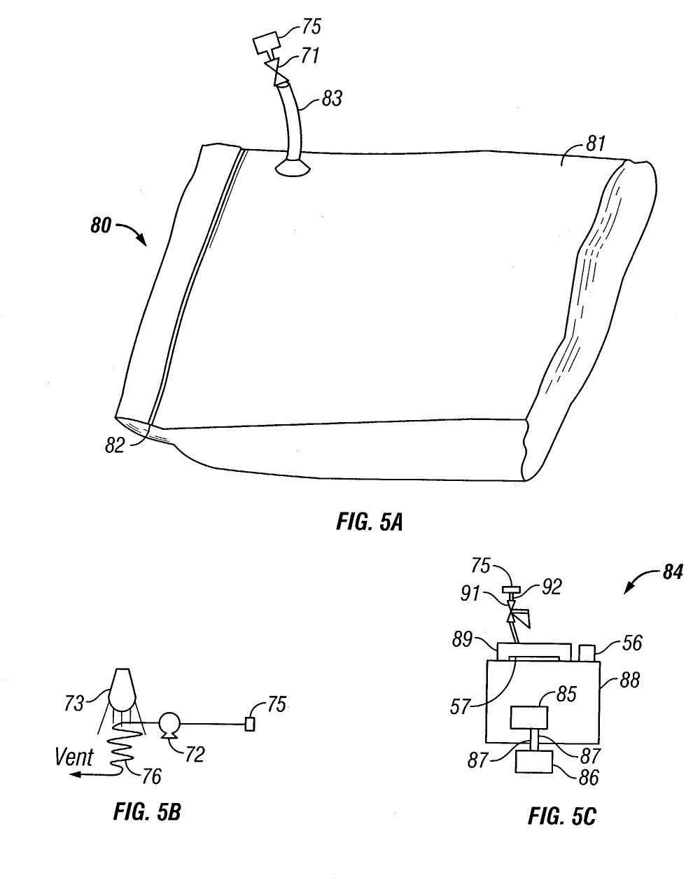

FIGS. 5A-C are perspective views of a medical instrument sterilizer having flexible walls in accordance with the present invention. While described as a medical instrument sterilizer, the invention is not to be so limited. The unit may be used, for example, to disinfect fresh produce (fruits and vegetables) by an army unit in the field. The sterilization chamber 81 is formed as a plastic bag, similar to commercially available vacuum bags, made of a nylon/polyethylene combination material, normally having a thickness of between about 2 mil and 7 mil. Nylon is preferably used in the bag construction to provide a longer vacuum life because without the nylon, air from the outside atmosphere will refill the bag in just a few hours. The sterilization chamber 81 may be sealed 82 with heat after the instruments to be sterilized are placed within the sterilization chamber 81. A tubing 83 in fluid communication with the interior of the sterilization chamber 81 may be sealed to the sterilization chamber 81. A valve 71 and quick connect fitting 75 at the end of the tubing 83 may be connected to a vacuum pump 72 for evacuating the sterilization chamber 81 after sealing the instruments inside. The sterilization chamber 81 may also be evacuated with the vacuum pump 72 after the sterilization process to purge the chamber 81 of the chlorine dioxide and to vent it to a safe location or to expose the chlorine dioxide flowing through a transparent tube 76 to a light source 73 to decompose the chlorine dioxide.

The sterilization unit 80 may include a separate electrolytic cell assembly 84 separated from the sterilization chamber 81. The electrolytic cell assembly 84 includes the electrode assembly 85, having the anode and the cathode, connected to a power supply 86 by conductors 87. The electrolyte reservoir 88 may be filled with the electrolytic aqueous solution or with the ingredients to form the electrolytic aqueous solution by being poured through the port having a removable cap 56 just as in the embodiment shown in FIG. 4. When power is applied across the electrodes in the electrode assembly 85, chlorine dioxide is generated and passes through the gas permeable membrane 57 into a chlorine dioxide chamber 89. A tubing 92 with a quick connect fitting 75 from the chlorine dioxide chamber 89 may be attached to the valve 71 on the sterilization chamber 81 to place the chlorine dioxide chamber 89 in fluid communication with the sterilization chamber 81. A pressure regulator 91 opens when sufficient pressure has developed in the chlorine dioxide chamber 89 to prevent the membrane 57 from being damaged by exposure to high pressure differential if the sterilization chamber 81 is under vacuum.

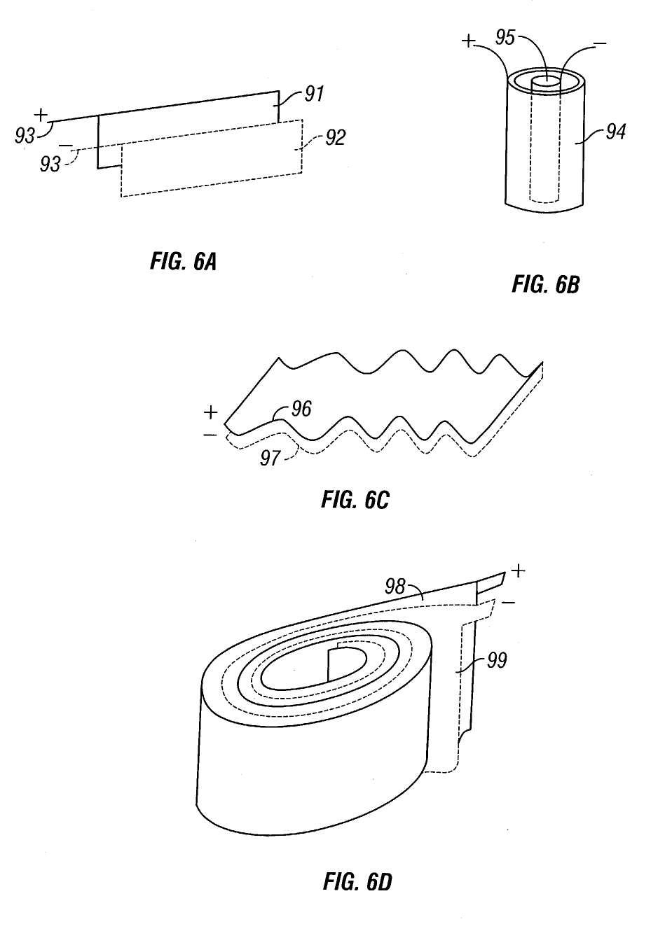

FIGS. 6 A-D are perspective views of examples of acceptable electrode assembly configurations in accordance with the present invention. In one alternative shown in FIG. 6A, both the cathode 92 and the anode 91 may be flat strips having wires 93 connected to the electrodes as leads to the power source. In FIG. 6B, the anode 94 may encircle the cathode 95 in a concentric arrangement. Alternatively, the cathode may encircle the anode in a concentric arrangement. In FIG. 6C, the cathode 97 and anode 96 may be in a folded arrangement, which provides additional surface area over straight strips or wires when the electrolyte reservoir limits the length of the electrode. In FIG. 6D, a Swiss-roll arrangement provides a cathode 99 and an anode 98, each formed from a flat strip, wound together in a spiral while still maintaining a constant gap between the faces of the anode 98 and the cathode 99.

### EXAMPLE 1

An electrochemical cell having cathode and anode areas of 5 cm×5 cm each was fabricated for conducting experiments on the generation of chlorine dioxide and sodium hypochlorite. The electrodes were made from porous titanium plates (Astromet Ti-ASTM B 265, pore size 25-40 microns and 0.065″ in thickness) made by the following procedure. Clean titanium frits were first platinized by electroplating, using Engelhard platinum salt A and the procedure recommended by Engelhard. These platinized titanium frits were used as the cathode and were used as the substrate for the anode, which was deposited with IrO₂ according to the following procedure. Iridium oxide was electroplated onto some frits in a solution formed by adding 0.416 g potassium hexachloroiridate (K₃IrCl₆), 0.5 g oxalic acid, and 2.76 g potassium carbonate to 200 mL DI H₂O, which was then kept for four days at 35° C. before plating at 0.4 mA/cm² for one hour per side. During the four-day hold period, the color of the coating changed from greenish, to brown, to clear to purple. The frits were then fired at 400° C. for one hour, and allowed to slowly cool before testing.

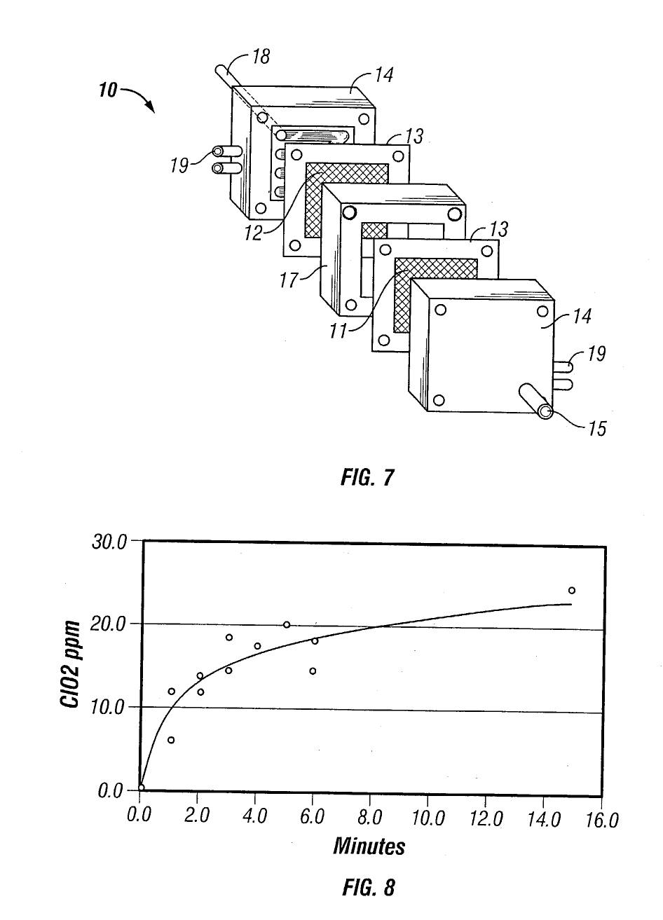

The electrochemical cell was assembled as shown in FIG. 7. The electrochemical cell 10 included the cathode 12 and the anode 11, both framed with a PTFE gasket 13. A plastic spacer 17 separated the two electrodes 11, 12. Two titanium endplates sealed the ends of the electrochemical cell 10. Deionized water containing sodium chloride and/or sodium chlorite was pumped through the electrochemical cell 10, entering the cell through the port 15 and exiting the cell through port 18 on the opposite end of the cell 10. Ports 19 were provided to circulate cooling water for cooling the cell 10. A power supply, not shown, was connected to provide voltage across the electrodes 11, 12.

A 750 mL salt solution containing 1.5 g/L NaCl and 1.5 g/L NaOCl₂ were pumped through the cell 10 at room temperature. The cell was run with a total current of 0.5 amps. Current density was maintained at about 20 mA/cm² and voltage was allowed to vary between about 2 Volts and about 4 Volts. Immediately upon application of voltage across the cell, gas evolution was observed and a gradual buildup of yellow color in the reservoir water was seen. The samples taken from the reservoir after 5 minute runs were analyzed. Samples were taken every five minutes and tested as follows. Sample solutions were titrated to test for free chlorine and chlorine dioxide with standard ferric ammonium sulfate titrant and N,N-diethyl-p-phenenediamine oxalate as stated in Standard Methods for the Examination of Water and Wastewater, 17^{th} ed., method 4500-Cl, Section F, and 4500-ClO₂, Section D. These methods were calibrated using method 4500-Cl Section G from the same source. The free chlorine, or hypochlorite, measurements include the sum total of hypochlorous acid (HOCl) and hypochlorite ion (OCl⁻) concentrations. The results are shown in FIG. 8. FIG. 8 shows the variation of ClO₂ concentration as a function of time.

### EXAMPLE 2

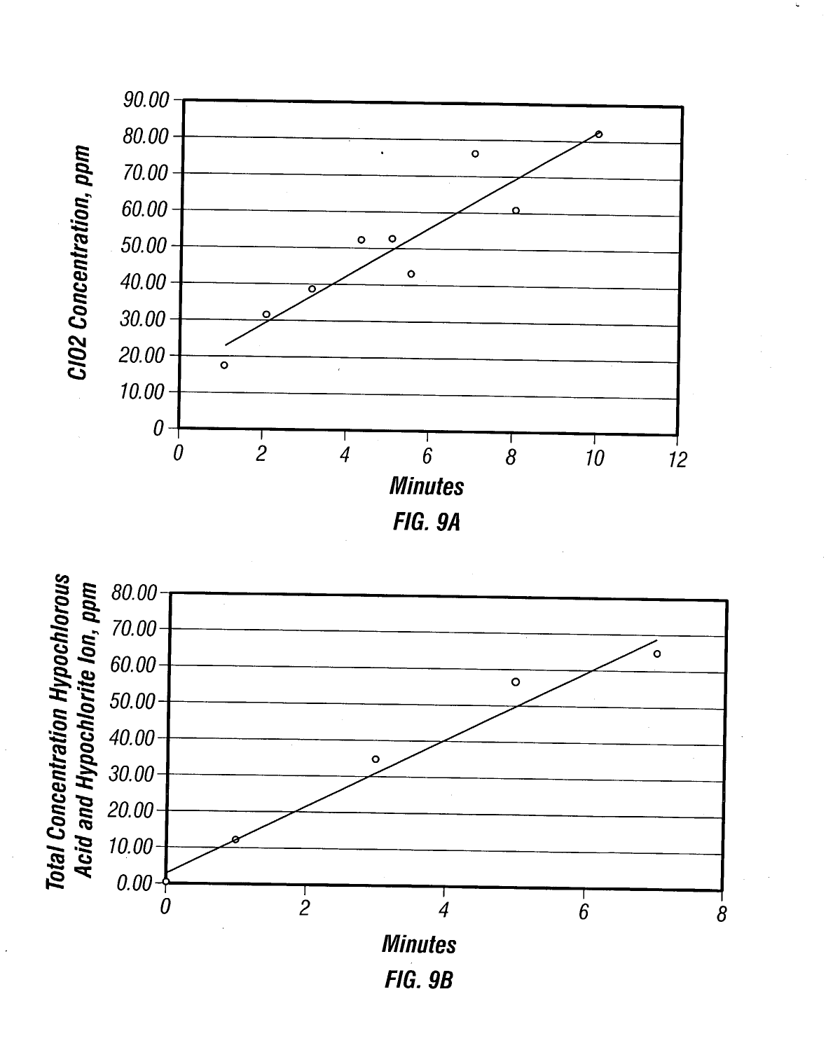

In this experiment, the same cell was used as in Example 1 except that the anode used in Example 1, the iridium dioxide coated 25 cm² frit, was replaced with a platinized titanium frit coated with ruthenium dioxide. RuO₂ was deposited according to the following procedure. Clean titanium frits were first platinized by electroplating as stated above. Ruthenium oxide was electroplated onto some frits in a solution formed by adding 0.416 g potassium hexachlororuthenate (K₃RuCl₆), 0.18 g anhydrous oxalic acid, and 1.38 g potassium carbonate to 100 mL DI H₂O, which was then kept for four days at 35° C. before plating at 0.4 mA/cm2 for one hour per side. During the four day hold period, the color of the coating changed from maroon, to green, to brown, and then to black. Coated frits were then fired at 400° C. for one hour, and allowed to slowly cool before testing. The cell was run with a solution containing 3 g/L sodium chlorite at a total current of 0.5 A and the variation of concentration of dissolved chlorine dioxide and the sum of the concentrations of hypochlorous acid and hypochlorite ion were measured. The results are shown in FIGS. 9A and 9B.

### EXAMPLE 3

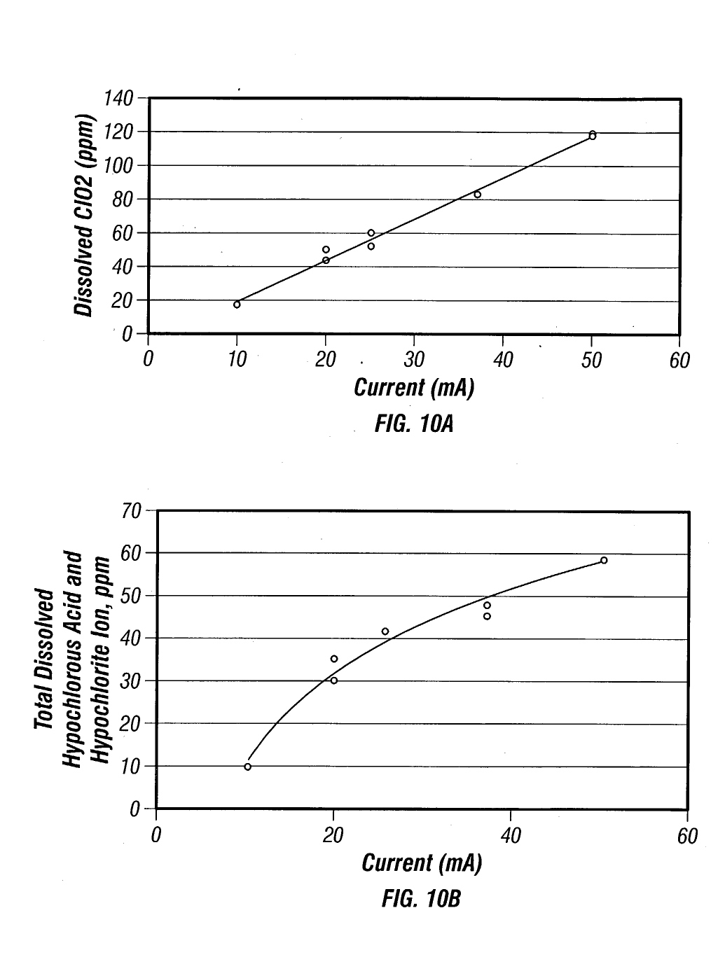

The generation of chlorine dioxide and hypochlorite was tested using a miniature platinized titanium frit (cathode) and a RuO₂ coated frit (anode), each having an area 1.23 cm². The frits were coated using the same procedure shown in Example 2. The solution used for electrolysis consisted of 1.5 g/L sodium chlorite and 1.5 g/L sodium chloride in deionized water. The electrolyses were conducted for a period of 2 minutes using 20 mL aliquots of the solution at different current densities. The variation of the concentration of dissolved chlorine dioxide as a function of total current is shown in FIG. 10A and the generation of dissolved hypochlorite is indicated in FIG. 10B. These data illustrate that the electrochemical cell can be easily scaled down while still producing the required concentration of chlorine dioxide. It is shown that an adequate volume of mouth rinse solution, about 20 mL, can be prepared using a scaled down version of the electrolytic cell of Example 1 in a period of under 2 minutes. It is known that a 10-20 ppm solution containing this dual disinfectant is more than sufficient to eliminate a high percentage of the bacteria that cause halitosis and dental plaque.

### EXAMPLE 4

Using the same miniature electrodes that were used in Example 3, the generation of chlorine dioxide and the sum of hypochlorous acid and OCl⁻ concentrations were measured. These experiments were conducted using a current of 20 mA, for 2 minutes in a solution of 20 mL volume. The results are shown in Table 1.

TABLE 1

| Sodium | Sodium | Chlorine | Hypochlorite ion and |
|---|---|---|---|
| chlorite, g/L | chloride, g/L | dioxide, ppm | hypochlorous acid, ppm |
| 3 | 0 | 51 | 30 |
| 1.5 | 1.5 | 47 | 33 |
| 1 | 2 | 28 | 14 |
| 0 | 3 | 0 | 34 |

The terms “comprising,” “including,” and “having,” as used herein, shall be considered as indicating an open group that may include other elements not specified. The terms “a,” “an,” and the singular forms of words shall be taken to include the plural form of the same words, such that the terms mean that one or more of something is provided. The term “one” shall be used to indicate that one and only one of something is intended. The terms “preferably,” “preferred,” and “may” are used to indicate that the item, condition or step being referred to is an optional (not required) feature of the invention.

It will be understood from the foregoing description that various modifications and changes may be made in the preferred embodiment of the present invention without departing from its true spirit. It is intended that this description is for purposes of illustration only and should not be construed in a limiting sense. Only the language of the following claims should limit the scope of this invention.

What is claimed is:

1. An apparatus, comprising:
a case;
an anode and a cathode, wherein the anode and the cathode are disposed for fluid communication with a reservoir;
an electrical power source for applying a voltage between the anode and the cathode, wherein the anode, the cathode and the electrical power source are contained within the case and wherein the reservoir contains an electrolyte.

2. The apparatus of claim 1, wherein the anode and the cathode are dimensionally stable electrodes.

3. The apparatus of claim 1, wherein the anode and the cathode comprise a metal.

4. The apparatus of claim 1, wherein the anode has a titanium substrate coated with an oxide selected from RuO₂, IrO₂ and combinations thereof.

5. The apparatus of claim 1, wherein the anode is made of a material selected from titanium, platinum, and combinations thereof.

6. The apparatus of claim 1, wherein the cathode has a titanium substrate coated with a metal.

7. The apparatus of claim 6, wherein the metal is selected from platinum, stainless steel, nickel, gold and combinations thereof.

8. The apparatus of claim 1, wherein the cathode is made of a material selected from titanium, platinum, graphite, steel, gold, iron, silver, tin and combinations thereof.

9. The apparatus of claim 1, wherein the electrolyte is an aqueous solution.

10. The apparatus of claim 1, wherein the electrolyte is an aqueous solution comprising metal chlorite.

11. The apparatus of claim 1, wherein the electrolyte is an aqueous solution having ingredients selected from the group consisting of sodium chlorite, sodium chloride, potassium chloride, potassium chlorite, an organic acid, a flavoring, a buffer and combinations thereof.

12. The apparatus of claim 11, wherein the organic acid is citric acid.

13. The apparatus of claim 1, wherein the electrolyte comprises metal chlorite having a concentration between about 0.01 g/L and about 6 g/L.

14. The apparatus of claim 1, wherein the electrolyte comprises metal chlorite having a concentration of between about 0.25 and about 1 g/L.

15. The apparatus of claim 1, wherein the electrolyte comprises metal chlorite having a concentration of between about 0.5 and about 0.6 g/L.

16. The apparatus of claim 1, wherein the electrolyte comprises metal chlorite and metal chloride and wherein the molar ratio of the metal chloride to the metal chlorite is between about 0.1 and about 10.

17. The apparatus of claim 1, wherein the electrolyte comprises metal chlorite and metal chloride and wherein the molar ratio of the metal chloride to the metal chlorite is between about 2 and about 9.

18. The apparatus of claim 1, wherein the electrolyte comprises metal chlorite and metal chloride and wherein the molar ratio of the metal chloride to the metal chlorite is between about 6 and about 8.

19. The apparatus of claim 1, wherein a distance of between about 0.3 and about 10 mm separates the anode and the cathode.

20. The apparatus of claim 1, wherein a distance of between about 0.5 and about 1 mm separates the anode and the cathode.

21. The apparatus of claim 1, wherein the cathode is a shape selected from a rod, a plate, a wire, a spiral, a Swiss-roll and combinations thereof.

22. The apparatus of claim 1, wherein the anode is a shape selected from a rod, a plate, a wire, a spiral, a Swiss-roll and combinations thereof.

23. The apparatus of claim 1, wherein the power source provides a voltage of between about 1.3 V to about 5 V.

24. The apparatus of claim 1, wherein the power source provides a voltage of between about 2 V to about 3 V.

25. The apparatus of claim 1, wherein the power source is one or more batteries.

26. The apparatus of claim 1, wherein the power source is a standard electrical outlet.

27. The apparatus of claim 1, wherein the current density supplied from the power source is between about 5 mA/cm² and about 100 mA/cm².

28.The apparatus of claim 1, wherein the current density supplied from the power source is between about 5 mA/cm² and about 30 mA/cm².

29. The apparatus of claim 1, wherein the current density supplied from the power source is between about 10 mA/cm² and about 20 mA/cm².

30. The apparatus of claim 1, further comprising a pellet storage compartment, wherein the pellets contain premeasured amounts of ingredients of the electrolyte.

31. The apparatus of claim 30, wherein the ingredients are selected from sodium chloride, sodium chlorite, potassium chloride, potassium chlorite, citric acid, a buffer, and combinations thereof.

32. The apparatus of claim 1, further comprising means for completing the circuit between the power source and the electrodes.

33. The apparatus of claim 1, wherein the anode and the cathode each measure between about 0.5 cm² and about 40 cm².

34. The apparatus of claim 1, wherein the anode and the cathode each measure between about 1 cm² and about 10 cm².

35. The apparatus of claim 1, wherein the anode and the cathode each measure between about 1 cm² and about 5 cm².

36. A personal mouthwash generator, comprising:
a case;
an anode and a cathode, wherein the anode and the cathode are disposed for fluid communication with a reservoir;
an electrical power source for applying a voltage between the anode and the cathode, wherein the anode, the cathode and the electrical power source are contained within the case, wherein the reservoir contains an electrolyte, wherein the electrolyte comprises a metal chlorite and wherein chlorine dioxide is produced by electrolytic reaction of the chlorite at the anode when voltage is applied across the anode and the cathode that are in fluid communication with the electrolyte.

37. The mouthwash generator of claim 36, wherein the anode and the cathode are dimensionally stable electrodes.

38. The mouthwash generator of claim 36, wherein the anode and the cathode comprise a metal.

39. The mouthwash generator of claim 36, wherein the anode has a titanium substrate coated with an oxide selected from RuO₂, IrO₂ and combinations thereof.

40. The mouthwash generator of claim 36, wherein the anode is made of a material selected from titanium, platinum, and combinations thereof.

41. The mouthwash generator of claim 36, wherein the cathode has a titanium substrate coated with a metal.

42. The mouthwash generator of claim 36, wherein the cathode is made of a material selected from titanium, platinum, graphite, steel, gold, iron, silver, tin, nickel and combinations thereof.

43. The mouthwash generator of claim 36, wherein the electrolyte is an aqueous solution comprising a metal chlorite.

44. The mouthwash generator of claim 36, wherein the electrolyte is an aqueous solution having ingredients selected from the group consisting of sodium chlorite, sodium chloride, potassium chlorite, potassium chloride, an organic acid, a flavoring, a buffer and combinations thereof.

45. The mouthwash generator of claim 44, wherein the organic acid is citric acid.

46. The mouthwash generator of claim 36, wherein the electrolyte comprises metal chlorite having a concentration between about 0.01 g/L and about 6 g/L.

47. The mouthwash generator of claim 36, wherein the electrolyte comprises metal chlorite having a concentration of between about 0.25 and about 1 g/L.

48. The mouthwash generator of claim 36, wherein the electrolyte comprises metal chlorite having a concentration of between about 0.5 and about 0.6 g/L.

49. The mouthwash generator of claim 36, wherein the electrolyte comprises metal chlorite and metal chloride and wherein the molar ratio of the metal chloride to the metal chlorite is between about 0.1 and 10.

50. The mouthwash generator of claim 36, wherein the electrolyte comprises metal chlorite and metal chloride and wherein the molar ratio of the metal chloride to the metal chlorite is between about 2 and 9.

51. The mouthwash generator of claim 36, wherein the electrolyte comprises metal chlorite and metal chloride and wherein the molar ratio of the metal chloride to the metal chlorite is between about 6 and 8.

52. The mouthwash generator of claim 36, wherein a distance of between about 0.3 and about 10 mm separates the anode and the cathode.

53.The mouthwash generator of claim 36, wherein a distance of between about 0.5 and about 1 mm separates the anode and the cathode.

54. The mouthwash generator of claim 36, wherein the power source provides a voltage of between about 1.3 V to about 5 V.

55. The mouthwash generator of claim 36, wherein the power source provides a voltage of between about 2 V to about 3 V.

56. The mouthwash generator of claim 36, wherein the current density supplied from the power source is between about 5 mA/cm² and 100 mA/cm².

57. The mouthwash generator of claim 36, wherein the current density supplied from the power source is between about 5 mA/cm² and 30 mA/cm².

58. The mouthwash generator of claim 36, wherein the current density supplied from the power source is between about 10 mA/cm² and 20 mA/cm².

59. The mouthwash generator of claim 36, further comprising a pellet storage compartment, wherein the pellets contain premeasured amounts of ingredients of the electrolyte.

60. The mouthwash generator of claim 36, wherein the ingredients are selected from sodium chloride, sodium chlorite, potassium chloride, potassium chlorite, citric acid, a buffer, and combinations thereof.

61. The mouthwash generator of claim 36, further comprising means for completing the circuit between the power source and the electrodes.

62. The mouthwash generator of claim 36, wherein the anode and the cathode each measure between about 1 cm² and about 10 cm².

63. The mouthwash generator of claim 36, wherein the anode and the cathode each measure between about 1 cm² and about 5 cm².

64. A mouth irrigator, comprising:
a reservoir for containing an electrolyte solution;
an anode and a cathode that are disposed for fluid communication with the reservoir;
an applicator for delivering the solution from the reservoir to a mouth; and
an electrical power source for applying a voltage between the anode and the cathode.

65. The mouth irrigator of claim 64, wherein the anode and the cathode are dimensionally stable electrodes.

66. The mouth irrigator of claim 64, wherein the anode and the cathode comprise a metal.

67. The mouth irrigator of claim 64, wherein the anode has a titanium substrate coated with an oxide selected from RuO₂, IrO₂, and combinations thereof.

68. The mouth irrigator of claim 64, wherein the anode is made of a material selected from titanium, platinum, and combinations thereof.

69. The mouth irrigator of claim 64, wherein the cathode has a titanium substrate coated with a metal.

70. The mouth irrigator of claim 64, wherein the cathode is made of a material selected from titanium, platinum, graphite, steel, gold, iron, silver, tin, nickle and combinations thereof.

71. The mouth irrigator of claim 64, wherein the electrolyte is an aqueous solution comprising a metal chlorite.

72. The mouth irrigator of claim 64, wherein the electrolyte is an aqueous solution having ingredients selected from the group consisting of sodium chlorite, sodium chloride, potassium chloride, potassium chlorite, an organic acid, a flavoring, a buffer and combinations thereof.

73. The mouth irrigator of claim 72, wherein the organic acid is citric acid.

74. The mouth irrigator of claim 64, wherein the electrolyte comprises metal chlorite having a concentration between about 0.01 g/L and about 6 g/L.

75. The mouth irrigator of claim 64, wherein the electrolyte comprises metal chlorite having a concentration of between about 0.25 and about 1 g/L.

76. The mouth irrigator of claim 64, wherein the electrolyte comprises metal chlorite having a concentration of between about 0.5 and about 0.6 g/L.

77. The mouth irrigator of claim 64, wherein the electrolyte comprises metal chlorite and metal chloride and wherein the molar ratio of the metal chloride to the metal chlorite is between about 0.1 and 10.

78. The mouth irrigator of claim 64, wherein the electrolyte comprises metal chlorite and metal chloride and wherein the molar ratio of the metal chloride to the metal chlorite is between about 2 and 9.

79. The mouth irrigator of claim 64, wherein the electrolyte comprises metal chlorite and metal chloride and wherein the molar ratio of the metal chloride to the metal chlorite is between about 6 and 8.

80. The mouth irrigator of claim 64, wherein a distance of between about 0.3 and about 10 mm separates the anode and the cathode.

81. The mouth irrigator of claim 64, wherein a distance of between about 0.5 and about 1 mm separates the anode and the cathode.

82. The mouth irrigator of claim 64, wherein the power source provides a voltage of between about 1.3 V to about 5 V.

83. The mouth irrigator of claim 64, wherein the power source provides a voltage of between about 2 V to about 3 V.

84. The mouth irrigator of claim 64, wherein the current density supplied from the power source is between about 5 mA/cm² and 100 mA/cm².

85. The mouth irrigator of claim 64, wherein the current density supplied from the power source is between about 5 mA/cm² and 30 mA/cm².

86. The mouth irrigator of claim 64, wherein the current density supplied from the power source is between about 10 mA/cm² and 20 mA/cm².

87. The mouth irrigator of claim 64, further comprising a pellet storage compartment, wherein the pellets contain premeasured amounts of ingredients of the electrolyte.

88. The mouth irrigator of claim 64, wherein the ingredients are selected from sodium chloride, sodium chlorite, potassium chloride, potassium chlorite, citric acid, a buffer, and combinations thereof.

89. The mouth irrigator of claim 64, further comprising means for completing the circuit between the power source and the electrodes.

90. The mouth irrigator of claim 64, wherein the anode and the cathode each measure between about 1 cm² and about 10 cm².

91. The mouth irrigator of claim 64, wherein the anode and the cathode each measure between about 1 cm² and about 5 cm².

92. A sterilizer, comprising:
a sterilization chamber;
an anode and a cathode that are disposed for fluid communication with an electrolyte chamber; wherein the electrolyte comprises a metal chlorite;
an electrical power source for applying voltage between the anode and the cathode to generate chlorine dioxide.

93. The sterilizer of claim 92, wherein the sterilization chamber and the electrolyte chamber are not separated.

94. The sterilizer of claim 92, wherein the sterilization chamber and the electrolyte chamber are separated by a gas permeable membrane.

95. The sterilizer of claim 95, wherein the gas permeable membrane is a material selected from PTFE (ethylene tetrafluoride resin), PFA (ethylene tetrafluoride-perfluoroalkoxyethylene copolymer resin), PVDF (vinylidene fluoride resin), FEP (ethylene tetrafluoride-propylene hexafluoride copolymer resin), ETFE (ethylene tetrafluoride-ethylene copolymer resin), and combinations thereof.

96. The sterilizer of claim 95, wherein the pore size of the gas permeable membrane is between about 0.01 μm and about 10 μm.

97. The sterilizer of claim 95, wherein the pore size of the gas permeable membrane is between about 0.1 μm and about 2 μm.

98. The sterilizer of claim 92, wherein the anode and the cathode are dimensionally stable electrodes.

99. The sterilizer of claim 92, wherein the anode and the cathode comprise a metal.

100. The sterilizer of claim 92, wherein the anode has a titanium substrate coated with an oxide selected from RuO₂, IrO₂, and combinations thereof.

101. The sterilizer of claim 92, wherein the anode is made of a material selected from titanium, platinum, and combinations thereof.

102. The sterilizer of claim 92, wherein the cathode has a titanium substrate coated with a metal.

103. The sterilizer of claim 92, wherein the cathode is made of a material selected from titanium, platinum, graphite, steel, gold, iron, silver, tin, nickel and combinations thereof.

104. The sterilizer of claim 92, wherein the electrolyte is an aqueous solution comprising a metal chlorite.

105. The sterilizer of claim 92, wherein the electrolyte is an aqueous solution comprising a sodium chlorite.

106. The sterilizer of claim 92, wherein the electrolyte is an aqueous solution having ingredients selected from the group consisting of sodium chlorite, sodium chloride, potassium chlorite, potassium chloride, an acid, a buffer and combinations thereof.

107. The sterilizer of claim 92, wherein the acid is citric acid.

108. The sterilizer of claim 92, wherein the electrolyte comprises metal chlorite having a concentration between about 0.01 g/L and about 6 g/L.

109. The sterilizer of claim 92, wherein the electrolyte comprises metal chlorite having a concentration of between about 0.25 and about 1 g/L.

110. The sterilizer of claim 92, wherein the electrolyte comprises metal chlorite having a concentration of between about 0.5 and about 0.6 g/L.

111. The sterilizer of claim 92, wherein the electrolyte comprises metal chlorite and metal chloride and wherein the molar ratio of the metal chloride to the metal chlorite is between about 0.1 and 10.

112. The sterilizer of claim 92, wherein the electrolyte comprises metal chlorite and metal chloride and wherein the molar ratio of the metal chloride to the metal chlorite is between about 2 and 9.

113. The sterilizer of claim 92, wherein the electrolyte comprises metal chlorite and metal chloride and wherein the molar ratio of the metal chloride to the metal chlorite is between about 6 and 8.

114. The sterilizer of claim 92, wherein a distance of between about 0.3 and about 10 mm separates the anode and the cathode.

115. The sterilizer of claim 92, wherein a distance of between about 0.5 and about 1 mm separates the anode and the cathode.

116. The sterilizer of claim 92, wherein the cathode is a shape selected from a rod, a plate, a wire, Swiss-roll and combinations thereof.

117. The sterilizer of claim 92, wherein the anode is a shape selected from a rod, a plate, a wire, Swiss-roll and combinations thereof.

118. The sterilizer of claim 92, wherein the anode and the cathode are spiral wound.

119. The sterilizer of claim 92, wherein the power source provides a voltage of between about 1.3 V to about 5 V.

120. The sterilizer of claim 92, wherein the power source provides a voltage of between about 2 V to about 3 V.

121. The sterilizer of claim 92, wherein the power source is one or more batteries.

122. The sterilizer of claim 92, wherein the power source is a standard electrical outlet.

123. The sterilizer of claim 92, wherein the current density supplied from the power source is between about 5 mA/cm² and 100 mA/cm².

124. The sterilizer of claim 92, wherein the current density supplied from the power source is between about 5 mA/cm² and 30 mA/cm².

125. The sterilizer of claim 92, wherein the current density supplied from the power source is between about 10 mA/cm² and 20 mA/cm².

126. The sterilizer of claim 92, further comprising a pellet storage compartment, wherein the pellets contain premeasured amounts of ingredients of the electrolyte.

127. The sterilizer of claim 92, wherein the ingredients are selected from sodium chloride, sodium chlorite, potassium chloride, potassium chlorite, citric acid, a buffer, and combinations thereof.

128. The sterilizer of claim 92, further comprising means for completing the circuit between the power source and the electrodes.

129. The sterilizer of claim 92, wherein the anode and the cathode each measure between about 0.5 cm² and about 40 cm².

130. The sterilizer of claim 92, wherein the anode and the cathode each measure between about 1 cm² and about 10 cm².

131. The sterilizer of claim 92, wherein the anode and the cathode each measure between about 1 cm² and about 5 cm².

132. A method of disinfecting, comprising:
preparing an aqueous solution including metal chlorite;
applying a voltage between an anode and a cathode that are within the aqueous solution to generate chlorine dioxide gas within the aqueous liquid and produce an aqueous solution comprising dissolved chlorine dioxide gas; and
contacting a contaminated surface with the aqueous chlorine dioxide-containing solution within 30 minutes after generating the chlorine dioxide gas.

133. The method of claim 132, further comprising;
dissolving metal chloride into the aqueous solution.

134. The method of claim 132, further comprising:
dissolving metal chloride, flavorings, a buffer, a sweetener, citric acid and combinations thereof into the aqueous solution.

135. The method of claim 132, further comprising: dissolving an organic acid into the aqueous solution, wherein the acid adjusts the pH level.

136. The method of claim 135, wherein the organic acid is citric acid.

137. The method of claim 132, further comprising:
dispensing a pellet comprising the metal chlorite into the aqueous solution.

138. The method of claim 132, further comprising:
dispensing a pellet comprising the sodium chlorite, sodium chloride, potassium chloride, potassium chlorite, a buffer, a sweetener, an acid and combinations thereof into the aqueous solution.

139. The method of claim 132, further comprising:
disposing the contaminated surface within the aqueous solution before the chlorine dioxide gas is generated.

140. The method of claim 132, further comprising:
disposing the contaminated surface in contact with the aqueous solution after the aqueous solution is no longer under an applied voltage.

141. The method of claim 132, wherein the anode and the cathode are dimensionally stable electrodes.

142. The method of claim 132, wherein the anode and the cathode comprise a metal.

143. The method of claim 132, wherein the anode has a titanium substrate coated with an oxide selected from RuO₂, IrO₂, PbO₂ and combinations thereof.

144. The method of claim 132, wherein the cathode is made of a material selected from titanium, platinum, graphite, steel, gold, iron, silver, tin and combinations thereof.

145. The method of claim 132, wherein the electrolyte comprises metal chlorite having a concentration between about 0.01 g/L and about 6 g/L.

146. The method of claim 132, wherein the electrolyte comprises metal chlorite having a concentration of between about 0.25 and about 1 g/L.

147. The method of claim 132, wherein the electrolyte comprises metal chlorite having a concentration of between about 0.5 and about 0.6 g/L.

148. The method of claim 132, wherein the electrolyte comprises metal chlorite and metal chloride and wherein the molar ratio of the metal chloride to the metal chlorite is between about 0.1 and 10.

149. The method of claim 132, wherein the electrolyte comprises metal chlorite and metal chloride and wherein the molar ratio of the metal chloride to the metal chlorite is between about 2 and 9.

150. The method of claim 132, wherein the electrolyte comprises metal chlorite and metal chloride and wherein the molar ratio of the metal chloride to the metal chlorite is between about 6 and 8.

151. The method of claim 132, wherein a distance of between about 0.3 and about 10 mm separates the anode and the cathode.

152. The method of claim 132, wherein a distance of between about 0.5 and about 1 mm separates the anode and the cathode.

153.The method of claim 132, wherein the cathode is a shape selected from a rod, a plate, a wire, a spiral and combinations thereof.

154. The method of claim 132, wherein the anode is a shape selected from a rod, a plate, a wire, a spiral and combinations thereof.

155. The method of claim 132, wherein the power source provides a voltage of between about 1.3 V to about 5 V.

156. The method of claim 132, wherein the power source provides a voltage of between about 2 V to about 3 V.

157. The method of claim 132, wherein the power source is one or more batteries.

158. The method of claim 132, wherein the power source is a standard electrical outlet.

159. The method of claim 132, wherein the current density supplied from the power source is between about 5 mA/cm² and 100 mA/cm².

160. the method of claim 132, wherein the current density supplied from the power source is between about 5 mA/cm² and 30 mA/cm².

161. The method of claim 132, wherein the current density supplied from the power source is between about 10 mA/cm² and 20 mA/cm².

162. The method of claim 132, wherein the anode and the cathode each measure between about 0.5 cm² and about 40 cm².

163. The method of claim 132, wherein the anode and the cathode each measure between about 1 cm² and about 10 cm².

164.The method of claim 132, wherein the anode and the cathode each measure between about 1 cm² and about 5 cm².

165. The method of claim 4, wherein the oxide coating may further be mixed with materials selected from cobalt, iron, bismuth and combinations thereof.

166. The mouthwash generator of claim 39, wherein the oxide coating may further be mixed with materials selected from cobalt, iron, bismuth and combinations thereof.

167. The mouth irrigator of claim 67, wherein the oxide coating may further be mixed with materials selected from cobalt, iron, bismuth and combinations thereof.

168. The sterilizer of claim 100, wherein the oxide coating may further be mixed with materials selected from cobalt, iron, bismuth and combinations thereof.

169. The method of claim 143, wherein the oxide coating may further be mixed with materials selected from cobalt, iron, bismuth and combinations thereof.

170. The sterilizer of claim 93, wherein walls of the sterilization chamber are constructed of materials selected from metal, rigid plastic and combinations thereof.

171. The sterilizer of claim 94, wherein the walls of the sterilization chamber are flexible.

172. The sterilizer of claim 171, wherein the walls are constructed of materials selected from plastic, nylon and combinations thereof.

173. The sterilizer of claim 94, further comprising means for placing the sterilization chamber in fluid communication with a vacuum source.

174.The sterilizer of claim 93, further comprising means for placing the sterilization chamber in fluid communication with a vacuum source.

175. A method for coating a substrate, comprising:
preparing an aqueous solution comprising materials selected from potassium hexachloroiridate, oxalic acid, calcium carbonate and combinations thereof;
storing the aqueous solution for a period, wherein the color of the aqueous solution changes from green, to brown, to clear, to purple during the period;
placing the substrate in the aqueous solution;
electroplating the iridium oxide onto the substrate.

176. The method of claim 175, wherein the aqueous solution is stored at a temperature between about 25° C. and about 45° C. during the period.

177. The method of claim 175, wherein the aqueous solution is stored at a temperature between about 30° C. and 40° C. during the period.

178. The method of claim 175, wherein a current density of between about 0.1 mA/cm2 and about 1 mA/cm² is applied during the electroplating step.

179. The method of claim 175, wherein a current density of less than 0.5 mA/cm2 is applied during the electroplating step.

180. The method of claim 175, wherein the concentration of potassium hexachloroiridate in the aqueous solution is between about 0.2 g and about 1 g per 100 ml of water.

181. The method of claim 175, wherein the concentration of potassium hexachloroiridate in the aqueous solution is between about 0.3 g and about 0.5 g per 100 ml of water.

182. The method of claim 175, wherein the concentration of oxalic acid is between about 0.1 g and about 5 g per 100 ml of water.

183. The method of claim 175, wherein the concentration of potassium carbonate is between about 0.1 g and about 5 g per 100 ml of water.

184. A method for coating a substrate, comprising:
preparing an aqueous solution comprising materials selected from hexachlororuthenate, oxalic acid, calcium carbonate and combinations thereof;
storing the aqueous solution for a period, wherein the color of the aqueous solution changes from maroon, to green, to brown, to black during the period;
placing the substrate in the aqueous solution;
electroplating the ruthenium oxide onto the substrate.

185. The method of claim 184, wherein the aqueous solution is stored at a temperature between about 25° C. and about 45° C. during the period.

186. The method of claim 184, wherein the aqueous solution is stored at a temperature between about 30° C. and 40° C. during the period.

187. The method of claim 184, wherein a current density of between about 0.1 mA/cm² and about 1 mA/cm² is applied during the electroplating step.

188. The method of claim 184, wherein a current density of less than 0.5 mA/cm2 is applied during the electroplating step.

189. The method of claim 184, wherein the concentration of potassium hexachlororuthenate in the aqueous solution is between about 0.2 g and about 1 g per 100 ml of water.

190.The method of claim 184, wherein the concentration of potassium hexachlororuthenate in the aqueous solution is between about 0.3 g and about 0.5 g per 100 ml of water.

191. The method of claim 184, wherein the concentration of oxalic acid is between about 0.1 g and about 5 g per 100 ml of water.

192. The method of claim 184, wherein the concentration of potassium carbonate is between about 0.1 g and about 5 g per 100 ml of water.
